(19) United States
(12) Patent Application Publication
(10) Pub. No.: US 2021/0007760 A1
(43) Pub. Date: Jan. 14, 2021

(54) SURGICAL SYSTEMS WITH SESNSING AND MACHINE LEARNING CAPABILITIES AND METHODS THEREOF

(71) Applicant: CAZE TECHNOLOGIES, Tustin, CA (US)

(72) Inventor: Carina R. Reisin, Tustin, CA (US)

(21) Appl. No.: 16/944,941

(22) Filed: Jul. 31, 2020

Related U.S. Application Data
(63) Continuation of application No. PCT/US2019/016434, filed on Feb. 1, 2019.
(60) Provisional application No. 62/760,657, filed on Nov. 13, 2018.
(60) Provisional application No. 62/626,040, filed on Feb. 3, 2018.

(51) Int. Cl. A61B 17/22; A61B 17/3207; A61B 17/34; G16H 40/63; G16H 40/40; G16H 70/20; G06N 20/00

(52) U.S. Cl. CPC A61B 17/2202; A61B 17/3207; A61B 17/3478; G16H 40/63; G16H 40/40; A61B 2090/064; G06N 20/00; A61B 2017/22038; A61B 2017/22021; A61B 2017/22079; A61B 2217/007; G16H 70/20

(57) ABSTRACT

Systems and methods for determining surgical system settings during a surgical procedure are disclosed. The surgical systems comprise of a control system, a means for tissue removal, sensing capabilities and machine learning application(s). The sensing capabilities and machine learning application(s) are configured to determine type and/or properties of the removed tissue and to predict preferred surgical settings for optimized removal and surgical outcomes. The learning machine application(s) communicates these preferred settings to a surgical control system.

### CROSS-REFERENCE TO RELATED APPLICATIONS

The present application is a continuation of International Patent Application No. PCT/US19/16434, filed Feb. 1, 2019, which claims priority to and the benefit of U.S. Provisional Application No. 62/760,657, filed on Nov. 13, 2018, and also to U.S. Provisional Application No. 62/626,040, filed Feb. 3, 2018, the disclosures of all of which are incorporated by reference herein in their entireties for all purposes

### TECHNICAL FIELD

The present disclosure relates to surgical systems and methods with sensing and machine learning capabilities. More specifically, the disclosure relates to surgical systems with real time sensing data and machine learning applications to determine best surgical parameters setting during the surgical procedure.

### BACKGROUND

Surgical systems are utilized for removal of different tissue structures from different parts of the body. There are numerous surgical procedures which require the removal of specific or selected portions of tissue of very delicate nature without damaging the surrounding or otherwise healthy tissue. Such procedures are frequently required in surgical procedures connected with, but not limited to, removal of blood clots formed in situ within the vascular system of the body and impeding blood flow (thrombectomy), transmyocardial revascularization (TMR) to treat angina (chest pain), removal of natural lens (cataract surgery), removal some or all of the vitreous humor from the eye (vitrectomy), removal of tumors, removal of polyps, and removal of damaged tissue due to inflammation such as for the treatment of tendonitis (where tendons that connect muscle to bone become inflamed).

While during these procedures, surgeons know the location of the treated tissue through direct visualization and/or through imaging techniques, a surgeon often has no knowledge or indication of the mechanical and/or physical properties and/or composition of the tissue to be removed. As such, the surgeon is typically forced to rely on experience to set up the surgical system to remove the tissue in question without knowing its mechanical and physical properties. Yet, different tissue types within the same surgical procedure category may require very different handling and thus different surgical system settings to maximize the chances of successful removal and procedure outcomes overall.

For example, there is a wide range of thrombus (blood vessel clot) types. Thromboembolism is a significant cause of morbidity (disease) and mortality (death), especially in adults. Therefore, thrombectomy, the interventional procedure of removing a blood clot (thrombus) from a blood vessel, is a life-saving procedure mostly performed in emergency situations. Traditionally thrombus was considered as ‘red’ or fibrin rich and ‘white’ or platelet-rich classically thought most likely to result from atherosclerotic plaques. However, this is now recognized to be an oversimplification of the vast range of different potential clot types, which have different physical properties, such as friction properties (‘stickiness’). Different clot types require very different handling and surgical system settings to achieve a successful and timely removal. Use of sub-optimal or wrong treatment in time critical procedures such as thrombectomy can result in fatal outcomes.

Therefore, there is a need for surgical systems with sensing capabilities combined with machine learning applications that can in real time identify the type of tissue under treatment. Furthermore, once the tissue (type and/or properties) is identified, the machine learning application(s) can determine the preferred/optimal settings for the surgical system and communicate these settings to the surgical control system. The control system can then suggest preferred setting to the surgeon and/or to automatically adjust system parameters during the procedure for optimized surgical outcomes and minimal procedure duration.

### BRIEF SUMMARY OF THE INVENTION

This summary and the following detailed description should be interpreted as complementary parts of an integrated disclosure, which parts may include redundant subject matter and/or supplemental subject matter. An omission in either section does not indicate priority or relative importance of any element described in the integrated application. Differences between the sections may include supplemental disclosures of alternative embodiments, additional details, or alternative descriptions of identical embodiments using different terminology, as should be apparent from the respective disclosures.

In accordance with the present disclosure, systems and methods are provided for determining tissue properties and/or type during a surgical procedure involving the removal of different tissue structures from different parts of the body. Further, the present disclosure describes a method for determining optimized surgical system settings for the removal of the tissue during the tissue removal procedure.

In some embodiments, the present disclosure may comprise ultrasonic surgical systems and methods for removing tissue structures, for example blood clots from any blood vessels, including, but not limited to, from small blood vessels of the brain during an ischemic stroke. The ultrasonic surgical systems and methods of the present invention may be particularly suitable for use in removing any type of clots, regardless of the thrombus type. Further, in some embodiments, the present disclosure may relate to the removal of clots from blood vessels while preventing the introduction of emboli into the blood stream during the removal procedure.

In some embodiments, the systems and methods of the present disclosure may comprise of an ultrasonic catheter having a needle with a cutter at its distal end. The cutter may be a continuous tip of the needle. The cutting tip of the needle may oscillate to establish a cutting action for fragmentation of the tissue structure, e.g., a clot. The oscillating nature of the needle may also induce cavitation near the tip of the needle causing emulsification of the tissue structure. The ultrasonic catheter may have a horn coupled to a transducer that is configured to convert alternating current into mechanical oscillation of the horn. The ultrasonic catheter may further include a needle that is attached to the horn (directly or indirectly). The needle may include a passage through which fragmented/emulsified tissue structure may be aspirated. The needle may be vibrated by oscillation of the horn. The needle vibration provides for cutting of tissue structure and/or inducing cavitation proximate the tip of the needle.

In some embodiments, a “sleeve” may be coaxially disposed about the needle, so as to define an annular passage between the needle and the “sleeve”, for introducing irrigation fluid and/or any pharmacological and/or anticoagulant drugs into the tissue structure (e.g., a clot) site.

In some embodiments, the ultrasonic catheter may include a guidewire. The guidewire having a deployable collapsed surgical (e.g., thrombectomy) assisting element disposed proximate to its distal end portion, is sized to pass longitudinally through the entire length of the catheter and the inner lumen of the needle, and to project distally from the distal end of the catheter/needle. The surgical assisting element has a first condition wherein the surgical assisting element is retracted/collapsed and a second condition wherein the surgical assisting element is expanded/open and spans almost the entire lumen of the vessel. The guidewire is advanced through the catheter to pierce and traverse the tissue structure (e.g., a clot) while the surgical assisting element is in its retracted/collapsed form. Once the guidewire transverses the tissue structure, the surgical assisting element is expanded/open and pulled back until it is in a close proximity to the distal end side of the tissue structure. In an application of thrombectomy, the expanded/open surgical assisting element prevents the introduction of emboli into the blood stream and improves clot fragmentation/emulsification efficacy during the clot removal procedure.

In some embodiments, the ultrasonic catheter system may further comprise a control system (which may also be referred to in this disclosure as system controller, or controller) having a console that includes an associated drive circuitry in connection with the transducer of the ultrasonic catheter. The control system may be configured to selectively adjust the operating (oscillating) frequency of the transducer and vary the operating frequency of the needle, to thereby increase or decrease the mechanical cutting performance and/or the cavitational-induced performance (ultrasound power). The ultrasonic catheter control system can also be configured to adjust the aspiration of the fragmented tissue structure (e.g., a clot) and/or to control the flow rate of the irrigation. The control and adjustment of each of these parameters separately or in any combination (ultrasound power, aspiration and irrigation) provide a wide range of optimized settings for the removal of the entire range of tissue types (e.g., thrombus types).

In some embodiments, the present disclosure may further comprise the incorporation of sensing capabilities to the surgical systems for the removal of different tissue structures from different parts of the body. In some embodiments, the present disclosure may include machine learning application(s) configured to determine properties and/or type of the tissue being removed based on the sensing of one or more parameters during the surgical removal of the tissue. In other embodiments, the present disclosure incorporates machine learning application(s) that is configured to determine one or more preferred system parameters setting based on tissue properties and/or type determination, for optimized surgical tissue removal and minimal procedure duration.

The machine learning application(s) referred to herein can use supervised, unsupervised or semi-supervised learning methods and algorithms. The machine learning method(s) can include, but not limited to, (Deep) Neural Network(s), Naïve Bayes, Decision Tree(s), Regression Tree(s), Gaussian Process Regression, Support Vector Regressor, Fuzzy c-Means, and/or Gaussian Mixture model(s).

The present disclosure also encompasses methods for sensing and monitoring one or more system parameters and suggesting and/or automatically-adjusting at least one system operational parameter. Sensing and monitoring can be done on one or more of machine parameters. The sensed parameters depend on the particulars of a given surgical system and its operational principle(s), such as, but not limited to, cutting, resection, aspiration, ultrasound, laser ablation, heat, and/or a combination of the thereof. In such surgical systems, the sensed parameters may include, but not limited to, cutting speed, ultrasound power, ultrasound frequency, ultrasound phase, ultrasound stroke, aspiration flow, vacuum level, irrigation flow, heat generation, heat dissipation, and others. Machine sensing parameters can be performed directly and/or indirectly by measuring one or more changes in, but not limited to, ultrasound characteristics (such as frequency, amplitude, phase, and/or stroke length), voltage, current, impedance, vacuum pump speed, pressure levels, suction level, irrigation flow, temperature, and/or optical reflectivity/transmissivity/absorbance/scattering, using internal system built-in controllers and/or by incorporation of one or more sensors to the system, such as, but not limited to, pressure sensors, flow sensors, optical sensors, accelerometers, displacement sensors and/or others.

In some embodiments, the system provided in accordance to this disclosure, can include one or more machine learning applications. The machine learning application(s) may be configured to determine the type of tissue and/or its properties based on one or more of the sensed parameter(s). The machine learning application(s) can be trained using experimental data and/or previous procedure data. The type of tissue determination can be done by the machine learning application(s) at any point in time during the surgical procedure. It can be done one or more times and/or continuously during the procedure using data representative of a snapshot in time and/or over an elapsed time during the procedure.

In some embodiments, the machine learning application(s) is configured to communicate with surgical system controller(s). Machine learning application(s) communicate to the system controller(s) the tissue type/properties and/or the predicted preferred system settings for one or more of the system parameters, based on tissue type/properties determination and prediction model(s). In still yet another aspect of the present disclosure, the system is configured to suggest the preferable system settings based on machine learning application(s) to the surgeon, or operator of the system, and/or is configured to automatically change the system settings based on machine learning application(s) output.

### BRIEF DESCRIPTION OF THE DRAWINGS

The accompanying drawings, which are incorporated in and constitute a part of the specification, are for illustrative purposes only of selected embodiments, serve to explain the principles of the invention. These drawings do not describe all possible implementations and are not intended to limit the scope of the present disclosure.

FIG. 1 shows a schematic illustration of an ultrasonic surgical system, according to various embodiments of the present disclosure.

FIG. 2A is a cross-sectional view of one embodiment of a distal end of the ultrasound catheter of FIG. 1.

FIG. 2B is a cross-sectional view of one embodiment of a distal end of the ultrasound catheter of FIG. 2A taken through line 2B-2B.

FIG. 3A is a cross-sectional view of a second embodiment of a distal end of the ultrasound catheter of FIG. 1.

FIG. 3B is a cross-sectional view of one embodiment of a distal end of the ultrasound catheter of FIG. 3A taken through line 3B-3B.

FIG. 4A is a cross-sectional view of a third embodiment of a distal end of the ultrasound catheter of FIG. 1.

FIG. 4B is a cross-sectional view of a proximal end and hub of the ultrasound catheter of FIG. 1 associated with the third embodiment of a distal end of FIG. 4A.

FIG. 5A is a cross-sectional view of a fourth embodiment of a distal end of the ultrasound catheter of FIG. 1.

FIG. 5B is a cross-sectional view of a proximal end and hub of the ultrasound catheter of FIG. 1 associated with the fourth embodiment of a distal end of FIG. 5A.

FIGS. 6A-6B are diagrams illustrating a generalized sequence of steps for the use of the ultrasonic surgical system within a blood vessel, according to one embodiment of the present disclosure.

FIG. 7A is a cross-sectional view of one embodiment of a distal end of the ultrasound catheter of FIG. 1 including one embodiment of a guidewire with a deployable surgical assisting element in a first condition, having a retracted/collapsed surgical assisting element disposed proximate to its distal end portion.

FIG. 7B is a cross-sectional view of one embodiment of a distal end of the ultrasound catheter of FIG. 1 including guidewire of FIG. 7A in a second condition having an expanded/open surgical assisting element disposed proximate to its distal end portion.

FIGS. 8A-8B are diagrams illustrating a generalized sequence of steps for the use of the ultrasonic surgical system within a blood vessel including an embodiment of a guidewire having a deployable surgical assisting element, according to one embodiment of the present disclosure.

FIGS. 9A-9B are diagrams illustrating another generalized sequence of steps for the use of the ultrasonic surgical system within a blood vessel including a second embodiment of a guidewire having a deployable surgical assisting element, according to one embodiment of the present disclosure.

FIG. 10 is an exemplary behavior chart of examples of surgical system parameters as a function of tissue hardness/density.

FIG. 11A is a simplified diagram of an exemplary implementation of the present disclosure using database(s) and/or lookup table(s) and/or threshold values, according to one embodiment of the present disclosure.

FIG. 11B is a simplified diagram of an exemplary implementation of the present disclosure using machine learning application(s), according to one embodiment of the present disclosure.

FIG. 12 illustrates an example of a predictive machine learning application, according to one embodiment of the present disclosure.

FIG. 13 shows an exemplary flow chart of operation of one embodiment of an intra-operative process for a surgical procedure using sensing capabilities and machine learning application(s).

FIG. 14 shows an exemplary flow chart of operation of one embodiment of an intra-operative process for a surgical procedure using sensing capabilities and machine learning application(s) with feedback loop.

FIG. 15A is an exemplary behavior chart of a thrombectomy system ultrasound parameters as a function of clot hardness.

FIG. 15B is an exemplary behavior chart of a thrombectomy system aspiration parameters as a function of clot hardness.

FIG. 16A is another simplified diagram of an exemplary implementation of the present disclosure using database(s) and/or lookup table(s) and/or threshold values for their use with the ultrasonic surgical system of FIG. 1, according to one embodiment of the present disclosure.

FIG. 16B is another simplified diagram of an exemplary implementation of the present disclosure using machine learning application(s) with the ultrasonic surgical system of FIG. 1, according to one embodiment of the present disclosure.

FIG. 17 illustrates an example of a predictive machine learning application for thrombectomy, according to one embodiment of the present disclosure.

FIG. 18 shows an exemplary flow chart of operation of one embodiment of an intra-operative process for a surgical procedure using sensing capabilities and machine learning application(s) for thrombectomy, according to one embodiment of the present disclosure.

FIG. 19 shows an exemplary flow chart of operation of one embodiment of an intra-operative process for a surgical procedure using sensing capabilities and machine learning application(s) with feedback loop for thrombectomy.

FIG. 20 shows a conceptual block diagram illustrating components of a system for determining surgical system settings during a surgical procedure, according to one embodiment of the present disclosure.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a”, “an”, and “the” are intended to include the plural forms as well as the singular forms, unless the context clearly indicates otherwise. It will be further understood that the terms “comprises” and/or “comprising”, when used in this description, specify the presence of stated features, steps, operations, elements, and/or components, but do not preclude the presence or addition of one or more other features, steps, operations, elements, components, and/or groups thereof.

As used herein, “occlusion,” “clot”, “blockage”, or “thromboembolism” refer to both complete and partial blockages of a vessel. Additionally, as used herein, “proximal” refers to that portion of the device or apparatus located closest to the user, and “distal” refers to that portion of the device or apparatus located furthest from the user. Additionally, as used herein, the term “catheter” is a broad term and is used in its ordinary sense and means, without limitation, an elongated flexible tube configured to be inserted into the body of a patient, such as, for example, a body cavity, duct or vessel.

The present disclosure is to be considered as an exemplification of the invention and is not intended to limit the invention to specific embodiments illustrated by the figures or description below. Individual elements or features of a particular embodiment are generally not limited to that particular embodiment, but, where applicable, are interchangeable and can be used in a selected embodiment, even if not specifically shown or described.

A number of different technologies are used for removal of different tissue structures from different parts of the body. Technologies used for tissue removal may include, but are not limited to, cutting, resection, aspiration, irrigation, ultrasound, laser, heat, and/or a combination of the thereof technologies. While during these procedures, surgeons know the location of the treated tissue through direct visualization and/or through imaging techniques, a surgeon often has no knowledge or indication of the mechanical and/or physical properties and/or composition of the tissue to be removed. As such, the surgeon is typically forced to rely on experience to set up the surgical system to remove the tissue in question without knowing its mechanical and physical properties. Yet, different tissue types within the same surgical procedure category, may require very different handling and surgical system settings to maximize the success of the removal and the procedure outcomes.

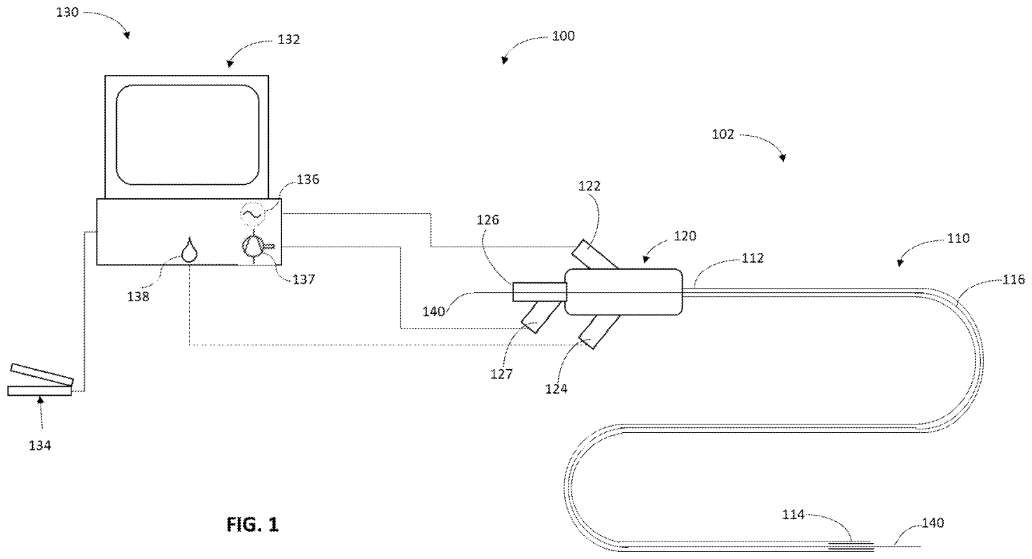

FIG. 1 illustrates an exemplary embodiment of a surgical system 100, comprising of an ultrasound catheter 102 and a control system 130. In an aspect, the surgical system 100 may be used in an embolectomy or thrombectomy procedure. The ultrasound catheter 102 generally comprises a multi-component tubular body 110 and a hub assembly 120. The tubular body 110 having a proximal end 112 and a distal end 114. The tubular body 110 and other components of the catheter 102 may be constructed from any number of suitable materials and techniques well known in the catheter manufacturing field. Catheter 102 may be dimensioned in any number of sizes and lengths depending upon, for example, entry point into the vasculature and location of a thromboembolism. Further, the tubular body 110 can be divided into a number of sections of varying stiffness. In one embodiment, the tubular body 110 can be divided into three sections. The first section, which may include the proximal end 112, may be generally stiffer than a second section between the proximal end 112 and the distal end 114 of the catheter. The third section 114, which may include the ultrasonic components and needle, may be generally stiffer than the second section due to the presence of these components. The ultrasound catheter 102 may also include a main inner lumen 116 extending between the proximal end of the hub 120, through a proximal end 112 and a distal end 114 of the tubular body 110.

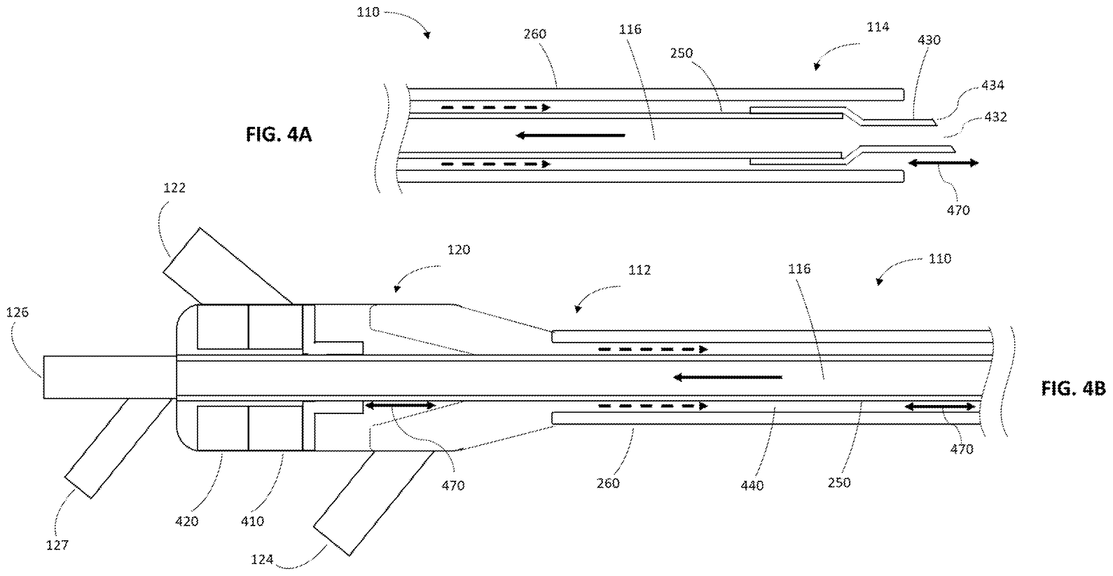

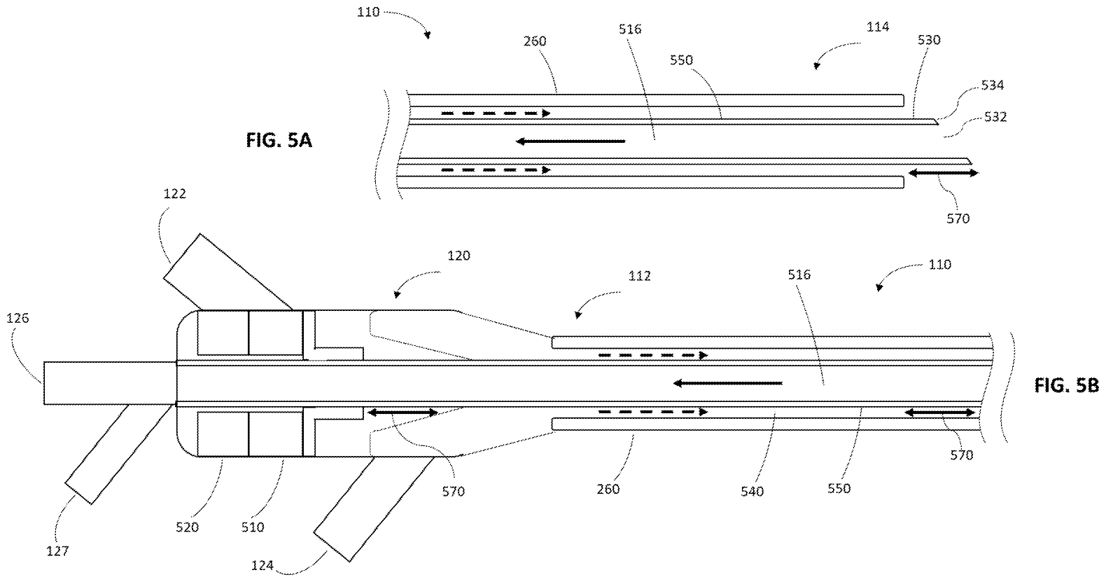

The hub assembly 120 may be coupled to the proximal end 112 for the purpose of coupling the tubular body 110 to the control system 130. The hub assembly 120 may also include a seal 126 for allowing the passage of a guidewire 140, an aspiration port 127, an irrigation port 124 and a drive circuitry port 122. In some embodiments, as described further in FIGS. 4A-5B, the hub assembly 120 may also include a transducer and a horn.

The surgical system 100 may further include a control system 130 comprising a console 132 having an associated drive circuitry 136 in connection with the transducer of the ultrasound catheter 102 located at the distal end 114 of the catheter or at the hub 120 (will be discussed below). The associated drive circuitry 136 may be in connection with a power source (not shown) and is configured to provide a variable frequency alternating current to drive or excite the transducer at a select operating frequency. The control system 130 may be configured to control the associated drive circuitry 136 to selectively adjust the operating frequency of the transducer, based, in part, on inputs to the control system 130. Thus, the control system 130 may be configured to vary the vibration of the ultrasonic catheter needle, to increase or decrease the mechanical cutting performance and/or the cavitational-induced performance of the needle located at the distal end 114 of the catheter 102 (will be discussed in detail below). The control system also may comprise a vacuum/aspiration pump 137 (such as a peristaltic and/or a venturi type of pump), and/or a means for delivering and controlling fluid irrigation 138. The control system 130 may further receive inputs from an operator, one or more applications including machine learning application(s) and/or from an external source, to permit selection of a specific operating frequency, aspiration and/or irrigation rates, for example. The control and adjustment of each one of these parameters (ultrasound power, aspiration and irrigation) separately or in any combination provides a means of wide range of optimized settings for the removal of a wide range of tissue types, e.g., thrombus types. The input may be provided by an input device which may comprise a keyboard or display device associated with the console 132, or a computing device internal and/or external to the system control, or a touch screen on the console, for example. The control system 130 may further include a foot pedal 134 used by an operator to activate and/or control the ultrasound catheter ultrasound power, aspiration and/or irrigation rates. Further, an operator may use the foot pedal 134 to provide input to the control circuit 130 for adjusting the operating frequency of the transducer connected to drive circuitry 136, to control aspiration by adjusting the pump 137, and/or for controlling the flow rate of fluids by adjusting the irrigation parameters 138.

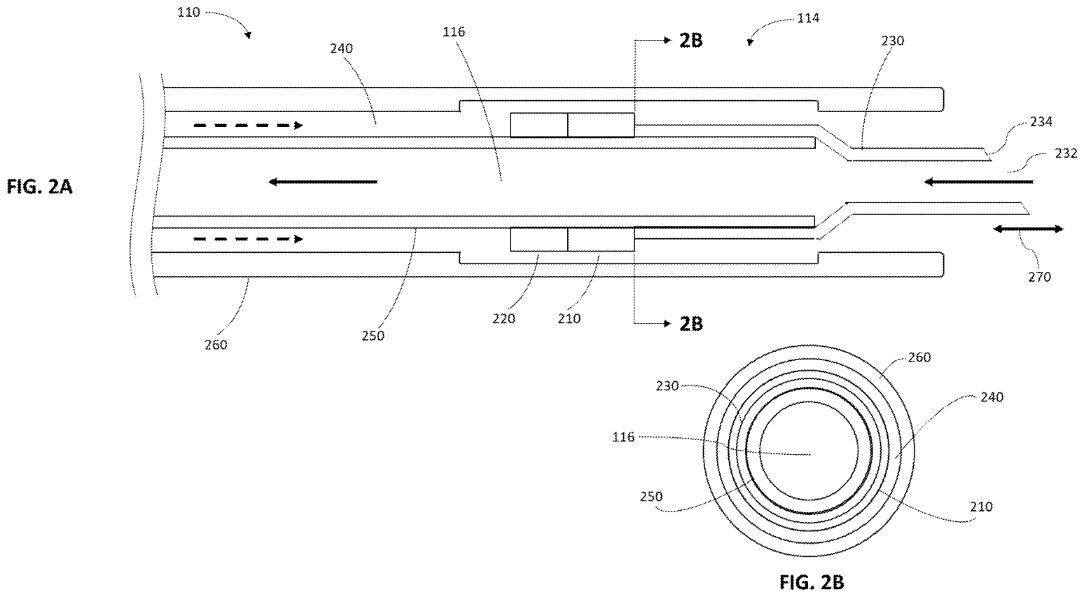

As shown in FIGS. 1 and 2A, the tubular body 110 may comprise an outer sheath 260 that is positioned upon an inner core 250. The distal end portion of the outer sheath 260 may be made of material and dimensions adapted for advancement through vessels. The inner core 250 may define, at least in part, an aspiration lumen 116, which extends longitudinally along the entire length of the catheter 102. The inner lumen 116 has a distal exit port 232 and a proximal access port 126. The tubular body 110 also having a space 240 formed between the outer sheath 260 and the inner core 250. Space 240 is connected to the hub assembly 120 and to an irrigation port 124 and/or to a drive circuitry port 122.

FIG. 2A illustrates one exemplary embodiment of the distal end 114 of an ultrasound catheter tubular body 110. FIG. 2B illustrates a cross-sectional view of the distal end 114 assembly of FIG. 2A as seen through line 2B-2B. In the illustrated embodiment, the distal end 114 having a horn 210 that may be mechanically coupled to one or more transducers 220, which convert high-frequency alternating current into mechanical vibrations. The horn 210 and transducer(s) 220 may be configured as hollow cylinders, such that the inner core 250 can extend through them. In this embodiment, the transducer(s) 220 may be coupled to the inner core 250, and the horn 210 may be coupled to the transducer(s) 220. These couplings may use adhesion or any mechanical attachment in a suitable manner. The horn 210 and transducer(s) 220 may take the form of solid rods, disks, or a plurality of smaller elements. The transducer 220 may be a magnetostrictive transducer, a microelectromechanical systems (MEMS) actuator, or a piezoelectric transducer for producing the vibrations or oscillations. The illustrated configuration provides an open irrigation path from the irrigation port 124 (FIG. 1) along the entire length of space 240. This open irrigation path may be used for introducing irrigation fluid and/or any pharmacological and/or anticoagulant drugs into the clot site and/or advantageously provide a cooling means for the ultrasound elements, where the irrigation fluid acts as a heat sink for removing heat generated by the elements.

The distal end 114 may further include a hollow needle 230 attached to the horn 210. The needle 230 may be vibrated by the mechanical oscillation of the horn 210 coupled to the transducer(s) 220. The mechanical vibrations of the horn 210 may rapidly move the needle tip 234 back and forth 270. This rapid movement of the needle tip provides a mechanical action, e.g., a jackhammer effect, causing a direct mechanical cutting or fragmentation, e.g., of the blockage upon contact with it. This rapid movement may also cause the radiation of ultrasonic energy into the surrounding tissue structure, e.g., a clot, and fluid that results in cavitational effects. Cavitation is defined as the growth, oscillation, and implosive collapse of micron sized bubbles in liquids under the influence of an acoustic field and may be created when the needle moves through a medium at ultrasonic speeds. When a cavitation bubble that forms can no longer sustain itself, the bubble or cavity implodes. The rapid cavitational collapse can produce shock waves and high-speed jets of liquid and can accelerate particles to high velocities. These effects can provide a mechanism for generating an impact against the surface of solids, where impingement of micro-jets and shock waves can create localized erosion of the surface. Thus, when the tip 234 of the needle 230 is brought into contact or close proximity of the occlusion, the occlusion material is disrupted in a jackhammer fashion by the mechanical cutting energy from needle 230, and/or the occlusion material is simultaneously emulsified by the implosion of cavitation bubbles generated from the rapid ultrasonic motion of the needle 230. The needle 230 may be made of any metals, ceramics, or plastics that may be suitable, for example, for intravascular thrombectomy. The needle tip 234 can be in a variety of configurations, including but not limited to, different bevel angles, bending angles and shapes.

The back and forth movement 270 of the needle tip 234 is defined as the stroke length or longitudinal excursion. The level of mechanical disruption and the level of cavitation induced emulsification are both defined by the stroke length associated with the operating frequency at which the needle 230 is vibrated. While the present example is directed to linear oscillation, the present disclosure may also be applied to torsional or transverse oscillation of the needle or any combination thereof.

Further, the distal end 114 of an ultrasound catheter tubular body 110 may have a passage 232 formed in the needle 230, horn 210 and along the entire tubular body 110, through which emulsified tissue structure and/or fluid may be aspirated.

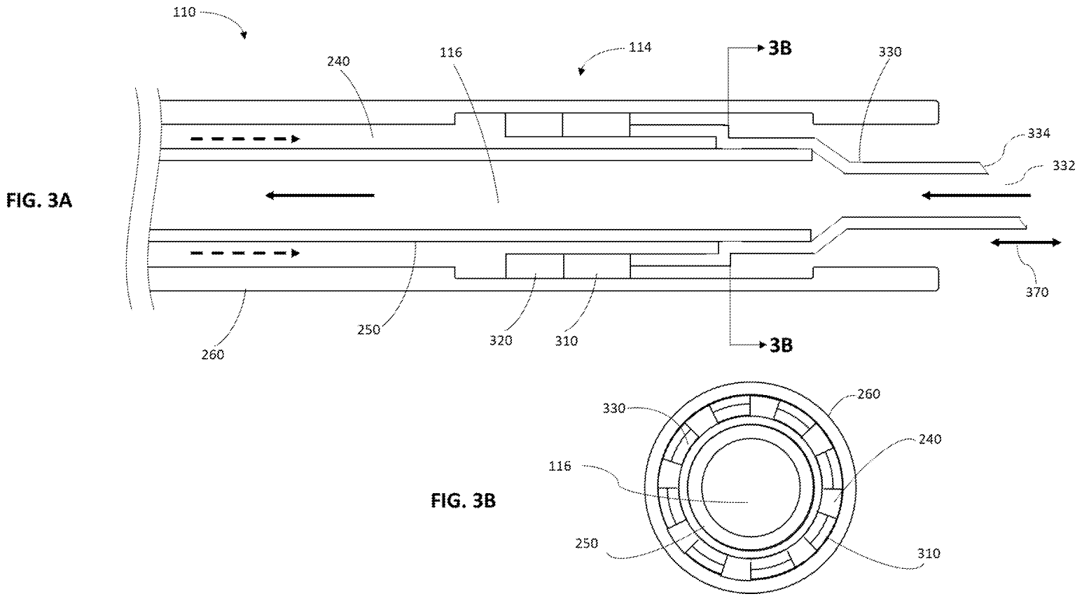

FIGS. 3A and 3B provide a second exemplary embodiment of the distal end 114 of an ultrasound catheter tubular body 110. FIG. 3B illustrates a cross-sectional view of the distal end 114 assembly of FIG. 3A as seen through line 3B-3B. In the illustrated second embodiment, the distal end 114 having one or more horns 310 that is/are mechanically coupled to one or more transducers 320, which convert high-frequency alternating current into mechanical vibrations. The horn 310 and transducer 320 are configured as hollow cylinders, such that the inner core 250 can extend through them. In this embodiment, the transducer(s) 320 may be coupled to the outer sheath 260, the horn 310 may be coupled to the transducer(s) 320, and the needle 330 may be coupled to the horn 310. These couplings may use adhesion or any mechanical attachment in a suitable manner. The horn 310 and transducer 320 may take the form of a plurality of small elements. The transducer(s) 320 may be a magnetostrictive transducer, a microelectromechanical systems (MEMS) actuator, or a piezoelectric transducer for producing the vibrations or oscillations. The illustrated configuration provides an open irrigation path from the irrigation port 124 (FIG. 1) along the entire length of space 240. This open irrigation path may be used for introducing irrigation fluid and/or any pharmacological and/or anticoagulant drugs into the clot site and/or advantageously provide a cooling means for the ultrasound elements, where the irrigation fluid acts as a heat sink for removing heat generated by the elements.

The distal end 114 may further include a hollow needle 330 attached to the horn(s) 310. The needle 330 may be vibrated by the mechanical oscillation of the horn(s) 310 coupled to the transducer(s) 320. The mechanical vibrations of the horn(s) 310 may rapidly move the needle tip 334 back and forth as shown in movement 370. This rapid movement of the needle tip provides a mechanical action, e.g., a jackhammer effect causing a direct mechanical cutting or fragmentation of a tissue structure, e.g., a clot, upon contact with it, and also causes the radiation of ultrasonic energy into the surrounding tissue structure and fluid that results in cavitational effects. Thus, when the tip 334 of the needle 330 is brought into contact or close proximity of the tissue structure, the tissue structure material is disrupted in a jackhammer fashion by the mechanical cutting energy from needle 330, and the tissue structure material is simultaneously emulsified by the implosion of cavitation bubbles generated from the rapid ultrasonic motion of the needle 330. The needle 330 may be made of any metals, ceramics, or plastics that may be suitable, for example for intravascular thrombectomy.

As in the previous described embodiment (FIGS. 2A and 2B), the back and forth movement 370 of the needle tip 334 is defined as the stroke length or longitudinal excursion. The level of mechanical disruption and the level of cavitation induced emulsification are both defined by the stroke length associated with the operating frequency at which the needle 330 is vibrated. While the present example is directed to linear oscillation, the present disclosure may also be applied to torsional or transverse oscillation of the needle or any combination thereof.

Some advantages of the surgical system as described in FIGS. 2 and 3 may include having the transducer(s) and horn coupled to the needle, thus providing a direct and/or proximal coupling/transfer of oscillation to the needle with minimal losses.

Further, the distal end 114 of an ultrasound catheter tubular body 110 may have a passage 332 formed in the needle 330 and along the entire inner lumen 116, through which emulsified tissue structure and/or fluid may be aspirated.

FIGS. 4A and 4B provide another exemplary embodiment of ultrasound catheter 102 (FIG. 1). FIG. 4A illustrates a third configuration of the distal end 114 of an ultrasound catheter tubular body 110, and FIG. 4B illustrates the proximal end 112 and the hub 120 of the ultrasound catheter associated with the embodiment of the distal end of FIG. 4A. In the illustrated FIG. 4B, the hub 120 having a horn 410 that is mechanically coupled to one or more transducers 420, which convert high-frequency alternating current into mechanical vibrations. The horn 410 and transducer(s) 420 are configured as hollow cylinders, such that the inner core 250 can extend through them. In this embodiment, the horn 410 and transducer(s) 420 may be coupled to the hub 120 and to inner core 250, and the horn 410 may be coupled to the transducer(s) 420. These couplings may use adhesion, or any mechanical attachment in a suitable manner. The horn 410 and transducer(s) 420 may take the form of solid rods, disks, or a plurality of smaller elements. The transducer 420 may be a magnetostrictive transducer, a microelectromechanical systems (MEMS) actuator, or a piezoelectric transducer for producing the vibrations or oscillations. The illustrated configuration provides an open irrigation path from the irrigation port 124 along the entire length of space 440.

The inner core 250 may be vibrated by the mechanical oscillation of the horn 410 coupled to the transducer(s) 420. The mechanical vibrations of the horn 410 may rapidly move the inner core 250 back and forth as shown in movement 470. The inner core may be composed of one or more radial layers and/or a range of durometers along the length of the tubing, such that it may have different mechanical properties along different axes to provide steering flexibility for catheter navigation through small vessels, and longitudinal strength to transmit the vibration and back and forth movement to the distal end of the inner core.

In this embodiment, the distal end 114 may include a hollow needle 430 attached to the end of the inner core 250 (FIG. 4A). The needle 430 may be vibrated by the mechanical oscillation of the inner core 250. The mechanical vibrations of the inner core 250 may rapidly move the needle tip 434 back and forth as shown in movement 470. Similar to the other described embodiments, this rapid movement of the needle tip provides a mechanical action, for example a jackhammer effect causing a direct mechanical cutting or fragmentation of a tissue structure upon contact with it, and also causes the radiation of ultrasonic energy into the surrounding tissue structure and fluid that results in cavitational effects. Further, as previously described, the distal end 114 of an ultrasound catheter tubular body 110 has a passage 432 formed in the needle 430 and along the entire inner lumen 116, through which emulsified tissue structure and/or fluid may be aspirated. The needle 430 may be made of any metals, ceramics, or plastics that may be suitable for, for example intravascular thrombectomy.

FIGS. 5A and 5B provide another exemplary embodiment of ultrasound catheter 102 (FIG. 1). FIG. 5A illustrates a fourth configuration of the distal end 114 of an ultrasound catheter tubular body 110, and FIG. 5B illustrates the proximal end 112 and the hub 120 of the ultrasound catheter associated with the embodiment of the distal end of FIG. 5A. In the illustrated FIG. 5B, the hub 120 having a horn 510 that may be mechanically coupled to one or more transducers 520, which convert high-frequency alternating current into mechanical vibrations. The horn 510 and transducer(s) 520 may be configured as hollow cylinders, such that the inner core 550 can extend through them. In this embodiment, the transducer(s) 520 may be coupled to the hub 120 and to inner core 550, and the horn 510 may be coupled to the transducer(s) 520. These couplings may use adhesion, or any mechanical attachment in a suitable manner. The horn 510 and transducer(s) 520 may take the form of solid rods, disks, or a plurality of smaller elements. The transducer 520 may be a magnetostrictive transducer, a microelectromechanical systems (MEMS) actuator, or a piezoelectric transducer for producing the vibrations or oscillations. The illustrated configuration provides an open irrigation path from the irrigation port 124 along the entire length of space 540.

The inner core 550 may be vibrated by the mechanical oscillation of the horn 510 coupled to the transducer(s) 520. The mechanical vibrations of the horn 510 may rapidly move the inner core 550 back and forth as shown in movement 570. The inner core in this embodiment is a hollow or a tube guidewire and might be composed of for example, but not limited to, solid steel and/or nitinol braided wire and/or nitinol tubes with micro-cut slots. Such inner cores should be designed with similar characteristics as guidewires in terms of pushability, steerability and torque to provide steering flexibility for catheter navigation through small vessels, and longitudinal strength to transmit the vibration and back and forth movement to the distal end of the inner core. The inner core might include markers (not shown) for visibility under imaging during the procedure.

In this embodiment, at the distal end 114 of the catheter the inner core end 530 may be configured with a “built in” needle tip 534 (FIG. 5A). The inner core needle tip can be of any configuration, including different angles and shapes. The mechanical vibrations of the inner core 550 may rapidly move the needle tip 534 back and forth as shown in movement 570. Similar to the other described embodiments, this rapid movement of the needle tip provides a mechanical action, for example a jackhammer effect, causing a direct mechanical cutting or fragmentation of a tissue structure upon contact with it, and also causes the radiation of ultrasonic energy into the surrounding tissue structure and fluid that results in cavitational effects. Further, as previously described, the distal end 114 of an ultrasound catheter tubular body 110 has a passage 532 formed at the distal end of the inner core 530 and along the entire inner lumen 516, through which emulsified tissue structure and/or fluid may be aspirated.

Some advantages of the surgical system as described in FIGS. 4 and 5 include the available space in the hub for the transducer(s) and the horn that can provide enough stroke movement and leave enough space for irrigation and aspiration, allowing for small diameter catheters, for example, where the outer diameter of the outer sheath is ≤2 mm and the inner core >1 mm.

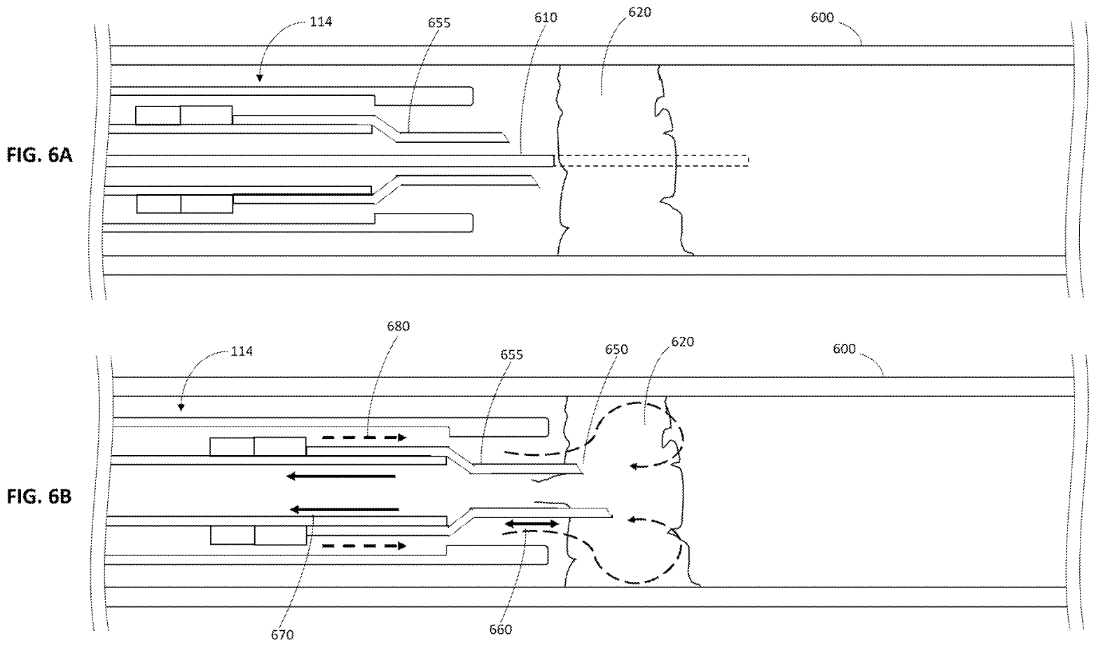

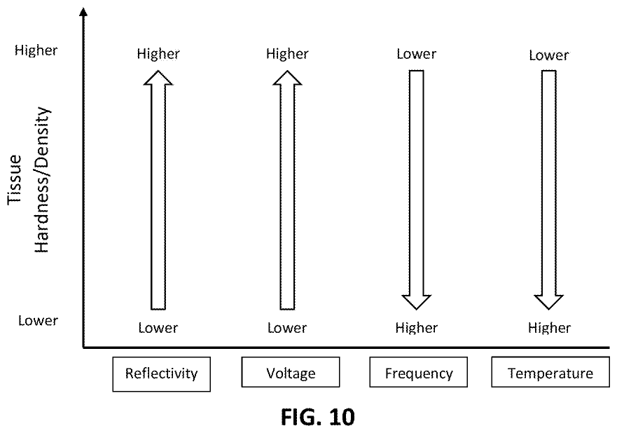

An exemplary method of using the surgical system 100 for embolectomy in connection with FIG. 1 will now be described with reference to FIGS. 6A and 6B. Although the following exemplary method is described using ultrasonic catheter configuration described in FIGS. 2A and 2B, this and similar methods of using the ultrasonic surgical system are applicable to other configurations of the ultrasonic catheter, such as those described in FIGS. 3A-5B. FIGS. 6A and 6B schematically depict a vessel 600 containing a blockage 620. In the first step, the ultrasound catheter 102 (FIG. 1) is introduced into the patient's vasculature (not shown). This process involves advancing a guidewire 610 to a point proximal to or to pierce and traverse the thromboembolism 620, as illustrated in FIG. 6A. The ultrasound catheter is then advanced over the guidewire to a point proximal to thromboembolism 620. The ultrasound catheter may have any markers, such as radiopaque marker band(s) encapsulated at the distal tip, for visualization under fluoroscopy (not shown). The surgical procedure is to position ultrasound catheter distal end 114, and more particularly the tip 650 of the needle 655, against and/or within the clot 620. Accordingly, as illustrated in FIG. 6B, the ultrasound catheter is advanced through vessel 600 until the distal end 114 is in contact with the clot 620 and the tip 650 of the needle 655 is positioned against and/or within the clot. At this point, the guidewire 610 may be retracted from the vessel or left in place. Once the distal end 114 is in contact with the clot 620 and the tip 650 of the needle 655 is positioned against and/or within the clot, the ultrasound power 660, aspiration 670, and/or irrigation 680 are activated, for example, by using the foot pedal 134 (FIG. 1). The tip of the needle 650 vibrates at ultrasonic frequency to disrupt and/or emulsify the clot while the aspiration pump aspirates particles through the tip and irrigation is employed to extract any potential heat buildup and/or to counteract any potential repulsive action of the ultrasonic needle 650. The operator/surgeon can prior to and/or during the procedure select any specific operating frequency, aspiration and/or irrigation rates, for example, and/or the settings selection can automatically be done by machine learning applications connected to the control system. The control and adjustment of each one of these parameters (ultrasound power, aspiration and irrigation) separately or in any combination provide a mean of wide range of optimized settings for the removal of different thrombus types. In some embodiments, as described in detail below (FIGS. 10-19), the ultrasonic surgical system may include technologies, including but not limited to sensors and machine learning applications, for selecting, controlling and adjusting of these parameters.

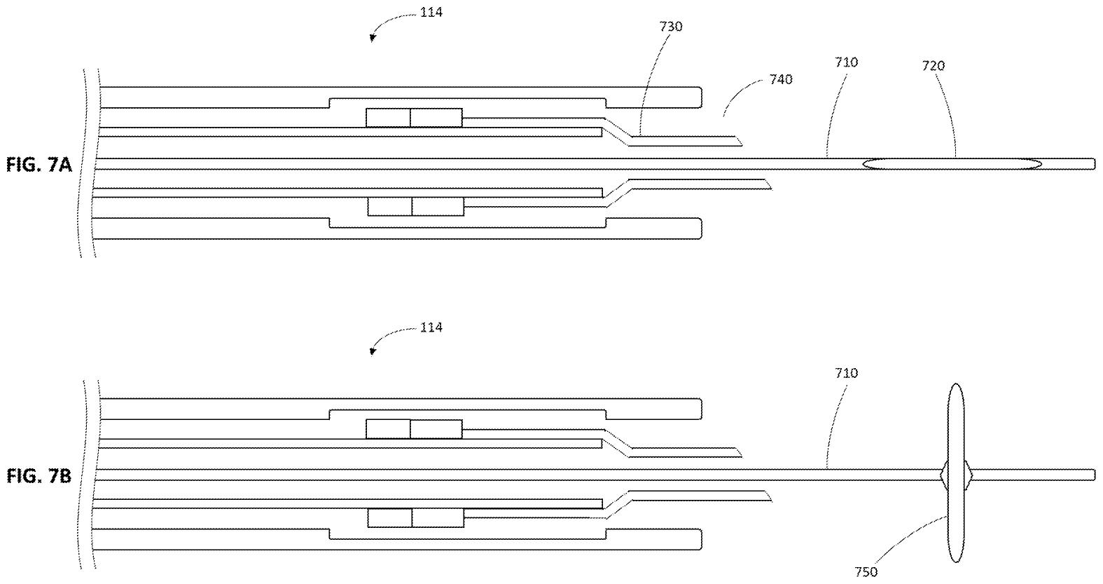

To further augment the ability of removing a thromboembolism while preventing the introduction of emboli into the blood steam and improving clot fragmentation/emulsification efficacy during the clot removal procedure, the ultrasonic catheter may include a guidewire having a deployable surgical assisting element disposed proximate to its distal end portion. In some embodiments, as illustrated in FIGS. 7A and 7B, the guidewire 710 having a collapsed surgical assisting element 720 disposed proximate to its distal end portion, is sized to pass longitudinally through the entire length of the catheter and the inner lumen of the needle 730 and to project distally from the distal end of the catheter/needle 740. The deployable surgical assisting element has a first condition wherein the surgical assisting element is retracted/collapsed 720 and a second condition wherein the surgical assisting element is expanded/open 750 and spans almost the entire lumen of the vessel (not shown).

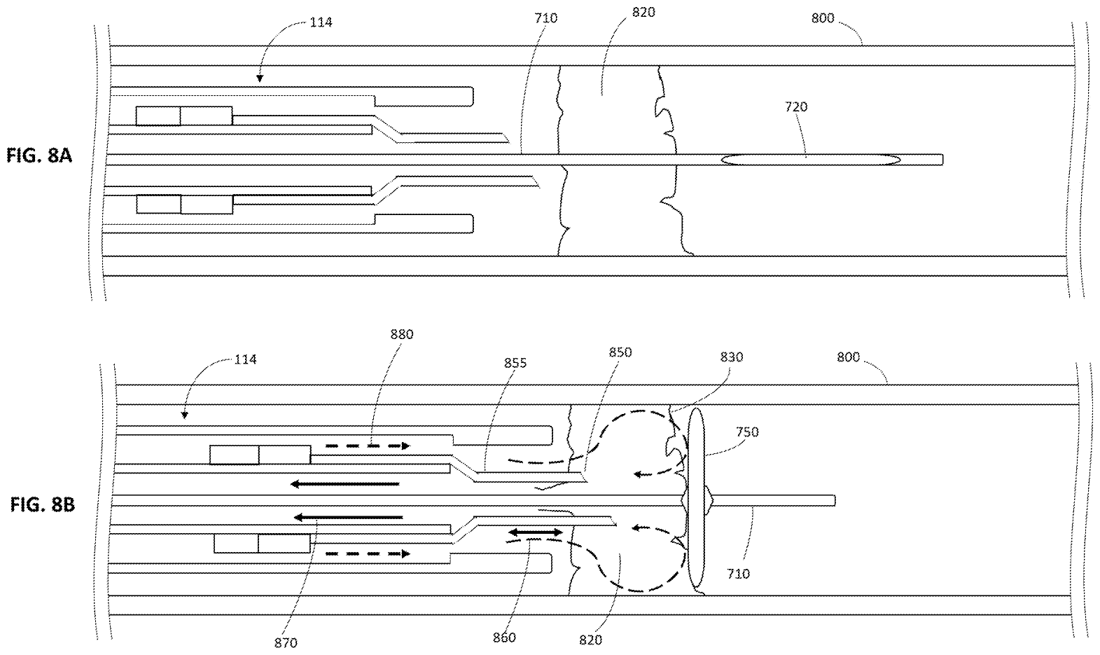

An exemplary method of embolectomy using guidewire 710 (FIGS. 7A and 7B) with the surgical system 100 (FIG. 1) will now be described with reference to FIGS. 8A and 8B. Although the following exemplary method is described using ultrasonic catheter configuration described in FIGS. 2A and 2B, this and similar methods of using the surgical system are applicable to other configurations of the ultrasonic catheter, such as those described in FIGS. 3A-5B. FIGS. 8A and 8B schematically show a vessel 800 containing a blockage 820. In the first step, the ultrasound catheter 102 (FIG. 1) is introduced into the patient's vasculature (not shown). This process involves advancing guidewire 710 through the vessel 800 to pierce and traverse the clot 820 while the surgical assisting element is in its retracted/collapsed form 720, as illustrated in FIG. 8A. The ultrasound catheter is then advanced over guidewire 710 to a point proximal to thromboembolism 820. The surgical procedure is to position ultrasound catheter distal end 114, and more particularly the tip 850 of the needle 855, against and/or within the clot 820. At this point, guidewire 710 surgical assisting element is expanded/open 750 and pulled back until it is in close proximity to the distal end side 830 of the clot 820. However, guidewire 710 surgical assisting element can also be expanded/open 750 and pulled back until it is in close proximity to the distal end side 830 of the clot 820 before the ultrasound catheter is advanced over guidewire 710 to a point proximal to thromboembolism 820. The guidewire may have any markers, such as radiopaque marker band(s) encapsulated in proximity to the surgical assisting element and/or at its distal end, for visualization under fluoroscopy (not shown).Once the distal end 114 is in contact with the clot 820, the needle tip 850 is positioned against and/or within the clot, and the expanded/open surgical assisting element 750 is in close proximity to the distal end side of the clot 830, the ultrasound power 860, aspiration 870, and/or irrigation 880 are activated. The expanded/open surgical assisting element 750 preventing the introduction of emboli into the blood steam and improves clot fragmentation/emulsification efficacy during the clot removal procedure by maintaining close proximity/contact between the clot 820 and the ultrasonic needle tip 850. At the end of the procedure, the guidewire surgical assisting element 750 can be collapsed before being extracted from the vessel.

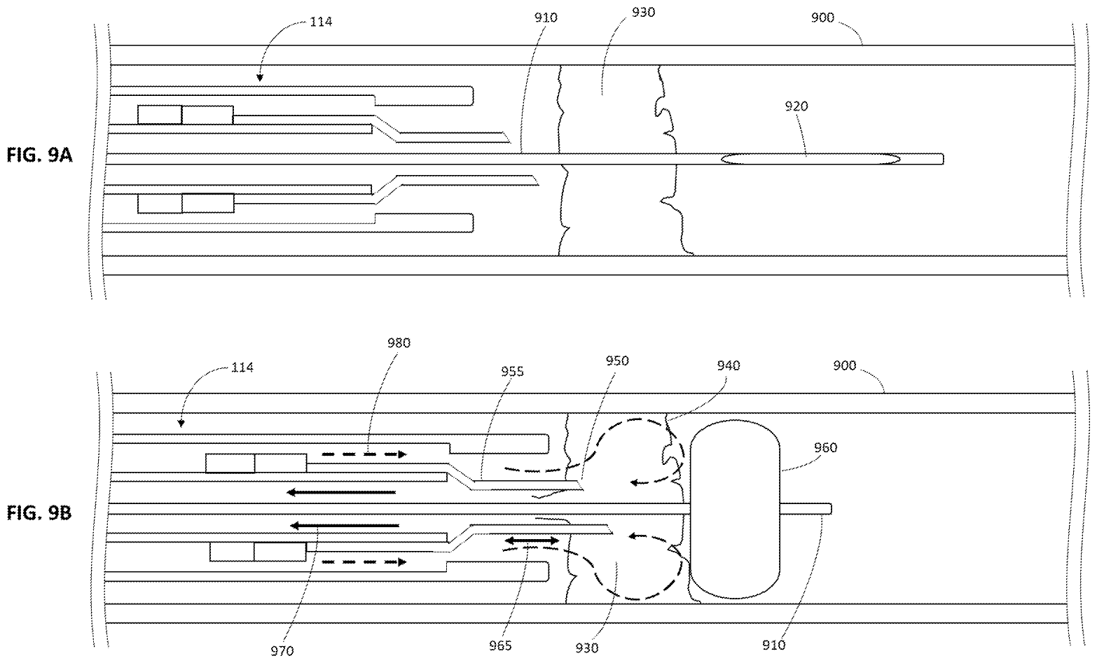

The surgical assisting element 720 (collapsed) and 750 (expanded/open) disposed proximate to the distal end portion guidewire 710 can be of many shapes and materials. Exemplary embodiments of the surgical assisting element shape are, but not limited to, a disc or pancake shaped element in its expanded/open condition 750 and formed of any suitable material, such as of metal or polymer, acting as a filter or a thrombectomy assisting element, or an expandable balloon as illustrated in FIGS. 9A and 9B. In this embodiment, the procedural process involves advancing guidewire 910 through the vessel 900 to pierce and traverse the clot 930 while the surgical assisting element/balloon is in its collapsed/uninflated form 920, as illustrated in FIG. 9A. The ultrasound catheter is then advanced over guidewire 910 to a point proximal to thromboembolism 930. The ultrasound catheter distal end 114 is position, and more particularly the tip 950 of the needle 955, against and/or within the clot 930 (FIG. 9B). At this point, guidewire 910 surgical assisting element/balloon is expanded/inflated 960 and pulled back until is in close proximity to the distal end side 940 of the clot 930. However, guidewire 910 surgical assisting element can also be expanded/inflated 960 and pulled back until it is in close proximity to the distal end side 940 of the clot 930 before the ultrasound catheter is advanced over guidewire 910 to a point proximal to thromboembolism 930. Once the distal end 114 is in contact with the clot 930, the needle tip 950 is positioned against and/or within the clot, and the expanded/inflated surgical assisting element/balloon 960 is in close proximity to the distal end side of the clot 940, the ultrasound power 965, aspiration 970, and/or irrigation 980 are activated. The expanded/inflated surgical assisting element/balloon 960 prevents the introduction of emboli into the blood steam and improvs clot fragmentation/emulsification efficacy during the clot removal procedure by maintaining close proximity/contact between the clot 930 and the ultrasonic needle tip 950. At the end of the procedure, the guidewire surgical assisting element 960 can be deflated before extracted from the vessel.

Although above exemplary embodiments of the surgical system 100 includes using ultrasonic technologies, the surgical system 100 may also include the use of at least one of resection-based technologies, laser-based technologies and heat-based technologies.

As mentioned above, in some embodiments, the surgical system and methods of the present disclosure may further include sensing and monitoring of surgical machine parameter(s) which can provide information on the type of tissue that has been removed at any time during the surgical procedure. The information provided on the type/properties of tissue by sensing and monitoring system parameter(s) may help operators, e.g., surgeons, to improve tissue removal, shorten surgical time and improve overall surgical outcomes.

The sensing and monitoring of machine parameter(s) at the beginning and/or during the surgical procedure of tissue removal can be done by monitoring the values of one or more parameters of the surgical system used. The sensed parameters depend on the surgical system and its operational principle(s), and therefore, the sensed parameter(s) depend on the particulars of the system and may include, but not limited to, cutting speed, ultrasound characteristics, aspiration, vacuum level, irrigation flow, heat generation/dissipation, optical properties and others. Machine parameter sensing can be performed directly and/or indirectly by measuring one or more changes in, but not limited to, ultrasound characteristics (such as frequency, amplitude, phase, and/or stroke length), voltage, current, impedance, vacuum pump speed, pressure levels, suction level, irrigation flow, temperature, and/or optical reflectivity/transmissivity/absorbance/scattering, using internal system built-in controllers and/or by incorporation of one or more sensors to the system, such as, but not limited to: pressure sensors, flow sensors, optical sensors, accelerometers, displacement sensors and/or others.

FIG. 10 is an exemplary illustrative graph showing the relationship between reflectivity (example as for laser-based technology), voltage (example as for ultrasound, and/or resection-based technologies), frequency (example as for ultrasound and laser-based technologies) and temperature (example as for heat and laser based technologies) as a function tissue hardness/density. A harder/denser tissue may cause higher reflectivity and voltage values, and manifest in lower resonant frequency and temperature values, while softer/sparser tissue will have the opposite effect.

Furthermore, based on the sensed and monitored parameters, the system may provide to the surgeon guidance with preferred machine settings to remove the tissue, and/or automatically modify system parameters with preferred machine settings by using database(s), lookup table(s) and/or machine learning application(s). To accurately map the behavior and the actual values of the sensed parameters as a function of tissue type/properties, experimental data or data from procedures may be obtained. This data will be used to generate database(s), look up table(s) and/or machine learning model(s). In particular, described in detail below are embodiments of surgical systems that utilize machine learning application(s) that is trained to learn, as an example, different sensed parameter values associated with tissue types/properties and determine preferred/optimized system parameters for tissue removal of the specific tissue under surgery.

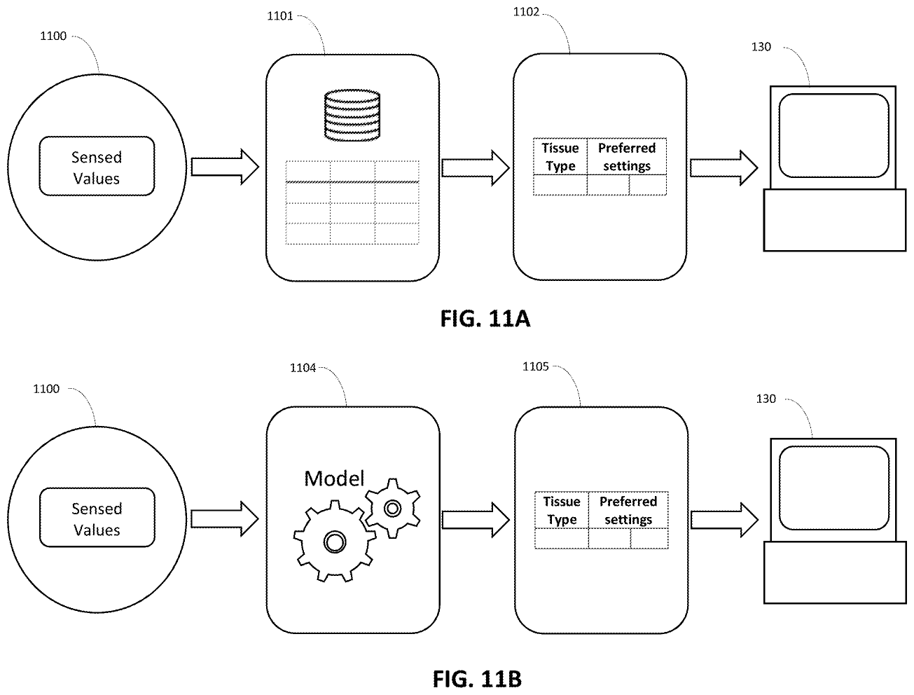

Referring to FIG. 11A, shown therein is an illustrative configuration of one embodiment of the disclosure. Sensed parameter values 1100 taken at any time during the procedure may be mapped into a database and/or lookup table 1101 that contains experimental data and/or data from previous procedures. Based on the data in the database and/or lookup table and/or defined threshold values, the system may find the best match between its data and received sensed values 1100 to define the tissue type/properties and/or preferred machine settings 1102. The identified preferred settings and/or tissue type/properties 1102 may then be communicated to the surgical control system 130. The surgical control system 130 may be programmed to notify the identified preferred settings and/or tissue type/properties to the surgeon and/or to automatically update the system parameters with the preferred settings 1102. While database(s), lookup table(s) and/or defined threshold values are relatively straight forward to generate and implement, they contain discrete data and are usually limited in their data scope and the number of parameters stored, thus making them less accurate.

In some embodiments, the surgical system for tissue removal may utilize machine learning application(s). Predictive machine learning application(s) uses algorithms to find patterns in data and then uses a model that recognizes those patterns to make predictions on new data. In this case, as illustrated in FIG. 11B, the sensed parameter values 1100 taken at any time during the procedure may be input into the machine learning application(s) 1104 that contain a machine learning model(s) generated based on experimental data and/or on data from surgical procedures. Based on the machine learning model(s), the machine learning application(s) predicts/determines the tissue type/properties and/or preferred machine settings 1105. The identified preferred setting and tissue type/properties 1105 may then be communicated to the surgical control system 130. The surgical control system 130 may be programmed to notify the identified preferred setting and/or tissue type/properties 1105 to the surgeon and/or to automatically update the system parameters with the predicted optimized/preferred settings 1105. The machine learning method(s) can include, but not limited to, (Deep) Neural Network(s), Naïve Bayes, Decision Tree(s), Regression Tree(s), Gaussian Process Regression, Support Vector Regressor, Fuzzy c-Means, and/or Gaussian Mixture model(s).

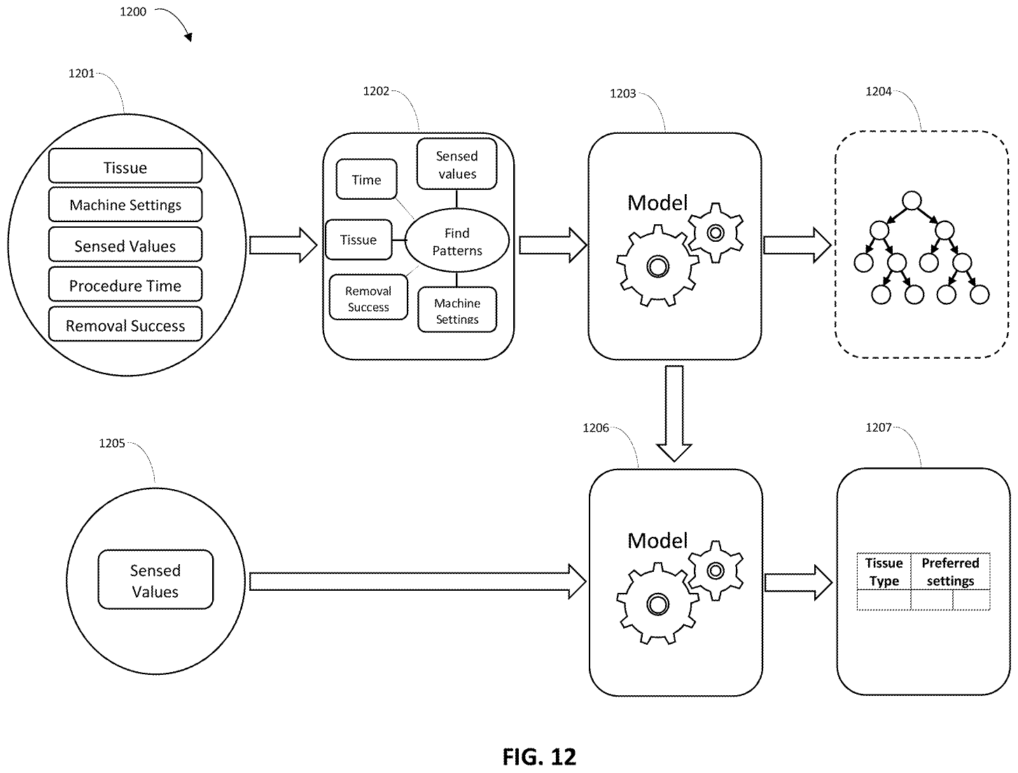

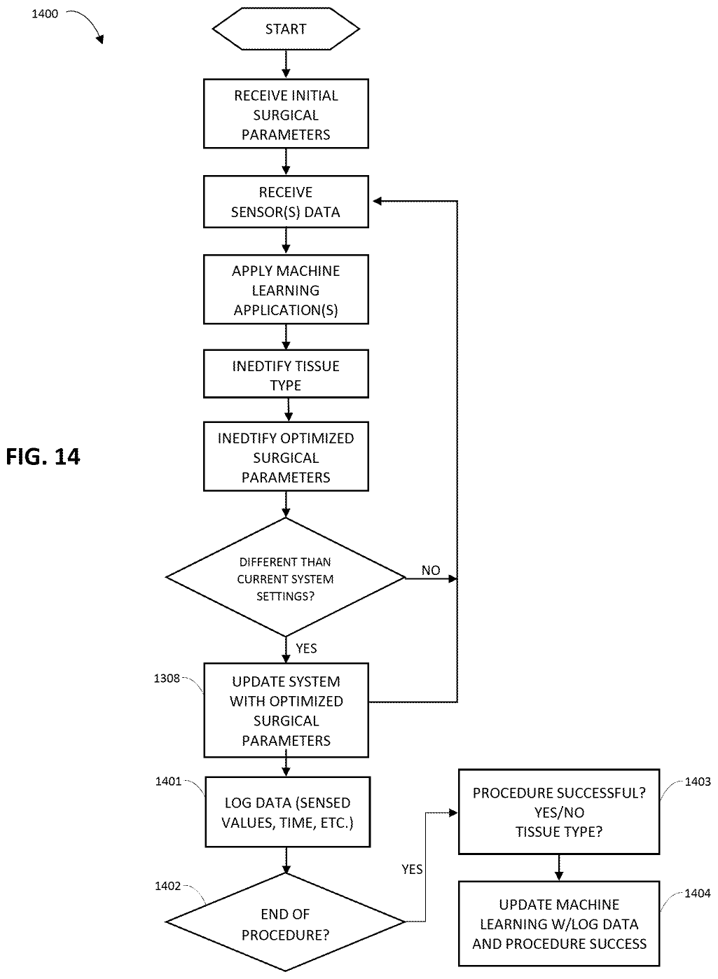

An example of a predictive machine learning application is illustrated in FIG. 12. The machine learning application 1200 is trained to learn, as an example, different parameters, procedure and machine setting values associated with tissue types/properties and determines preferred/optimized system parameters for the removal of the specific tissue. In this particular example, the machine learning application receives information 1201 on, but not limited to, tissue types/properties, machine settings, sensed values, procedure time and/or removal success. The data can be experimental data and/or data from previous procedures. A machine learning training algorithm 1202 may be developed to train the machine learning to find patterns within the provided data. Based on the identified patterns, a machine learning model 1203 may be developed from which a pattern(s) database 1204 can be generated. With any new surgical procedure, the sensed values during the procedure 1205 are input onto the machine learning application(s), and the application(s) uses the built model(s) 1206 to predict tissue type/properties and optimized/preferred machine settings 1207. In some embodiments, at the end of each procedure, the new collected data can be entered back into the machine learning application(s) to improve model(s)/predictability (FIG. 14). The machine learning application(s) can reside within a computing device internal and/or external to the system control, and one or more internal and/or external computing devices can be used to train the machine learning algorithm(s).

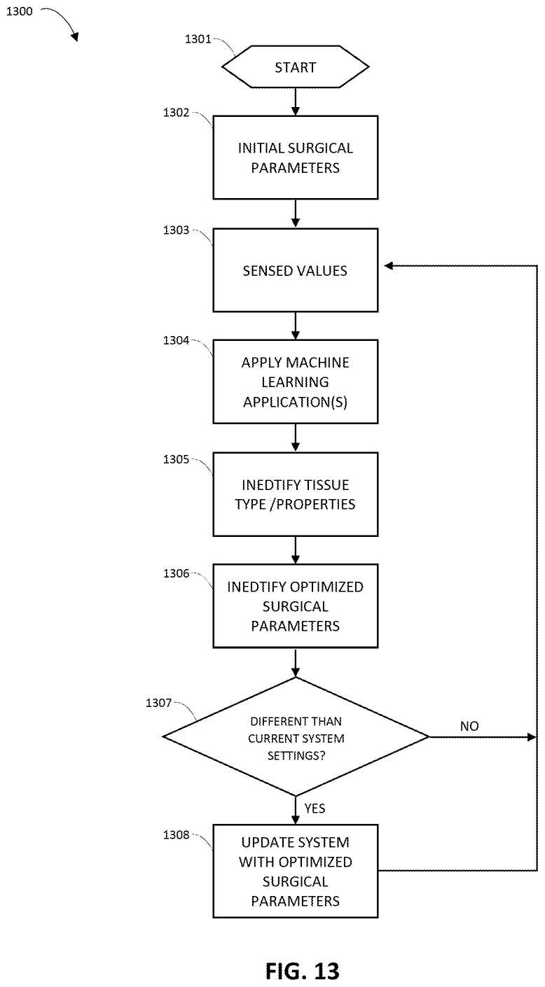

FIG. 13 is an exemplary flow chart 1300 describing a method for conducting a surgical procedure that includes machine learning application(s). At the beginning of each new procedure 1301, the surgical system control is set by internal algorithms and/or external source(s) (e.g. surgical staff, remotely via WiFi, etc.) with initial parameters at 1302. The input may be provided by an input device, which may comprise a keyboard or display device associated with the surgical control system 1103 (FIG. 11B), or a touch screen on the console, or an external source, for example. The surgical control system may further include a foot pedal and/or remote-control device used by an operator to activate and/or set machine parameters. Once the procedure starts and the surgical system/machine is set with initial settings and activated by any suitable means, the different defined and programmed sensed parameters may be sensed at 1303 and their values communicated to the machine learning application(s) at 1304. The machine learning application(s) determines the tissue type/properties at 1305 and preferred/optimized system settings at 1306 for the identified type of tissue. The optimized setting(s) can include one or more values related to any of the machine parameters, based on their technology. The predicted optimized parameters are compared to current machine setting at 1307. If any of these values differ, the system may be automatically updated with new settings at 1308. In any case, the process 1303 to 1308 may repeat iteratively for as long as the procedure continues. This process can be executed continuously and/or at defined periods/intervals of time.

Further, a method for conducting a surgical procedure for tissue removal that includes machine learning application(s) can also include processes where collected surgical data can be entered back into the machine learning application(s) to improve model(s)/predictability. An exemplary embodiment is shown in the flow chart described in FIG. 14. With reference to FIG. 14, every time the system surgical parameters are updated at 1308, surgical data such as, but not limited to, sensed values and time, can be logged into a database/table at 1401. At the end of each procedure at 1402 or at a later stage, the surgeon may specify whether the procedure was successful at 1403. Successful procedure can be described by yes/no, and/or by a defined scale from for example 1 to 10, and/or by any other criteria. Type/properties of tissue removed during the surgical procedure can also be collected at the end of the surgery at 1403. Tissue type/properties can be defined for example by a defined scale/score and/or by pathology examination of removed tissue. Following 1403, the log data at 1401 and procedure success data and/or tissue type/properties at 1403 may be input back into the machine learning trained data and can be used to continue improvement of its model and predictions.

Without limiting the scope of this disclosure, a specific example of one implementation of systems and methods of this disclosure in a thrombectomy application will now be described in detail.

In this thrombectomy application, sensing and monitoring machine parameter(s) can provide information on the type of clot being removed at any time during the surgical procedure. The techniques described herein may provide the surgeon with clot information and surgical settings which are critical for the revascularization procedure and improve success of clot removal. The information provided on the type of clot by sensing and monitoring system parameter(s) may help surgeons to improve clot removal, surgical time and overall revascularization outcomes. Further, based on the sensed parameters, the system may provide to the surgeon guidance with preferred machine settings to remove the clot, and/or, in some embodiments, by automatically modifying system parameters with optimized machine settings, by using data base(s), look up table(s) and/or machine learning application(s) as this is an urgent and timely procedure. In particular, as described in detail below, the surgical system 100 (FIG. 1) may utilize machine learning application(s) which is trained to learn, for example, different sensing parameter values associated with clot types and predict preferred/optimized system parameters for revascularization and removal of the specific clot.

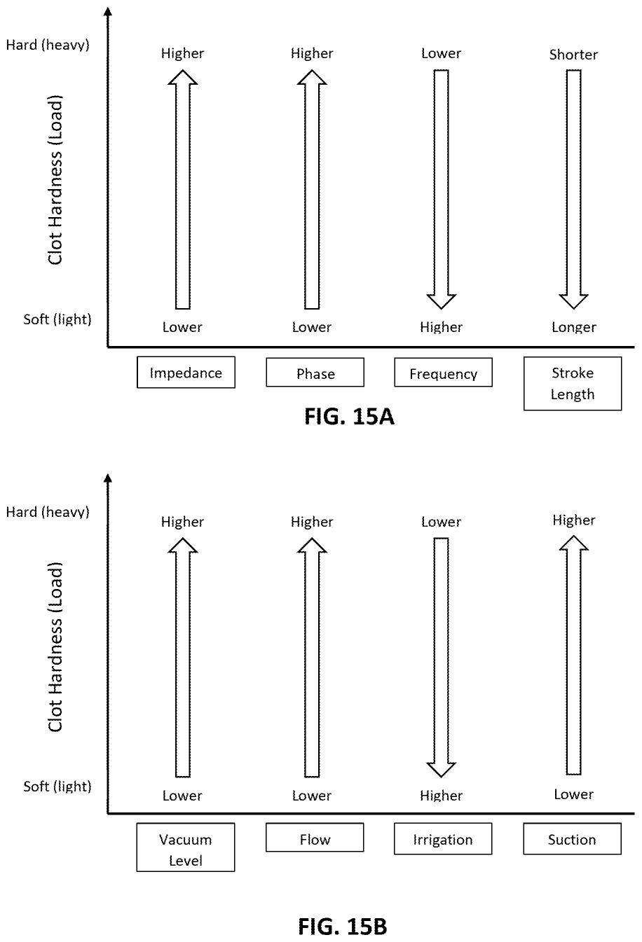

The sensing and monitoring of machine parameter(s) at the beginning and/or during the thrombectomy procedure, can be done by monitoring the values of one or more parameters of the surgical system 100, and include, but not limited to, ultrasound power, ultrasound frequency, ultrasound phase, ultrasound stroke, aspiration flow, vacuum level, irrigation flow, and others. Machine parameters sensing can be performed by measuring directly and/or changes in one or more parameters such as, but not limited to, ultrasound characteristics (such as frequency, amplitude, phase, mechanical load, impedance, voltage, current, and/or stroke length), vacuum pump speed, pressure levels, suction level, and/or irrigation flow, using internal machine built-in controllers/electronics and/or by incorporation of one or more sensors to the system, such as, but not limited to, pressure sensors, flow sensors, accelerometers, displacement sensors and/or others. FIG. 15A is an exemplary illustrative graph showing the relationship between ultrasound associated parameters such as impedance, phase, resonant frequency and stroke length as a function clot hardness. A harder clot (higher load to the ultrasound system) will cause higher impedance and phase values and shift the resonant frequency and stroke length to lower values. Similarly, FIG. 15B is an exemplary illustrative graph showing the relationship between vacuum parameters/irrigation such as vacuum level, flow, irrigation, and suction as a function clot hardness. A harder clot will cause higher vacuum levels, higher flow and higher suction, and will lower irrigation flow. Experimental and/or actual procedural data can be used to formulate the actual values of these parameters as a function clot type. The data formulating clot type can serve as a footprint of the clot being treated.

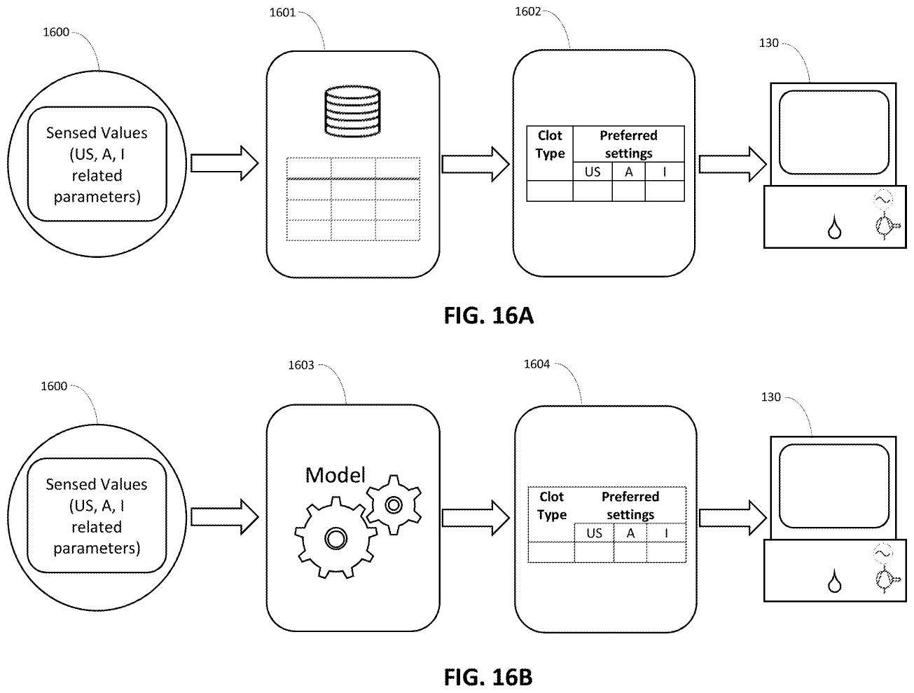

As described, based on the sensed parameters, the system may provide to the surgeon guidance with preferred machine settings to remove the clot, and/or, in some embodiments, can automatically modifying system parameters with optimized machine settings, by using database(s), look up table(s) and/or machine learning application(s). Referring to FIG. 16A, shown therein is an illustrative configuration of one embodiment of the disclosure. Sensed parameters values 1600 associated with ultrasound (US) and/or Aspiration (A) and/or Irrigation (I) parameters (as described above), taken at any time during the procedure are mapped into a database and/or lookup table 1601 that contain experimental data or data from previous procedures. Based on the data in the database and/or lookup table, and/or defined threshold values, the system finds the best match within its data and received sensed values 1600 to define the clot type and/or preferred machine settings 1602. The identified preferred setting and clot type 1602 are then communicated to the control system 130. Control system 130 may be programmed to notify the identified preferred setting and/or clot type to the surgeon and/or to automatically update one or more of the system parameters (ultrasound, aspiration and/or irrigation) with the preferred settings 1602.

In some embodiments, the surgical system 100 (FIG. 1) may utilize machine learning application(s) in an embolectomy application. In this case, as illustrated in FIG. 16B, the sensed parameter values 1600 taken at any time during the procedure are input into the machine learning application(s) 1603 that contain a machine learning model. Based on machine learning model, the machine learning application(s) defines the clot type and/or preferred machine settings 1604. The identified preferred setting and clot type (ultrasound, aspiration and/or irrigation) 1604 are then communicated to the control system 130. The control system 130 may be programmed to notify the identified preferred setting and/or clot type to the surgeon and/or advantageously to automatically update the system parameters with the preferred settings 1604.

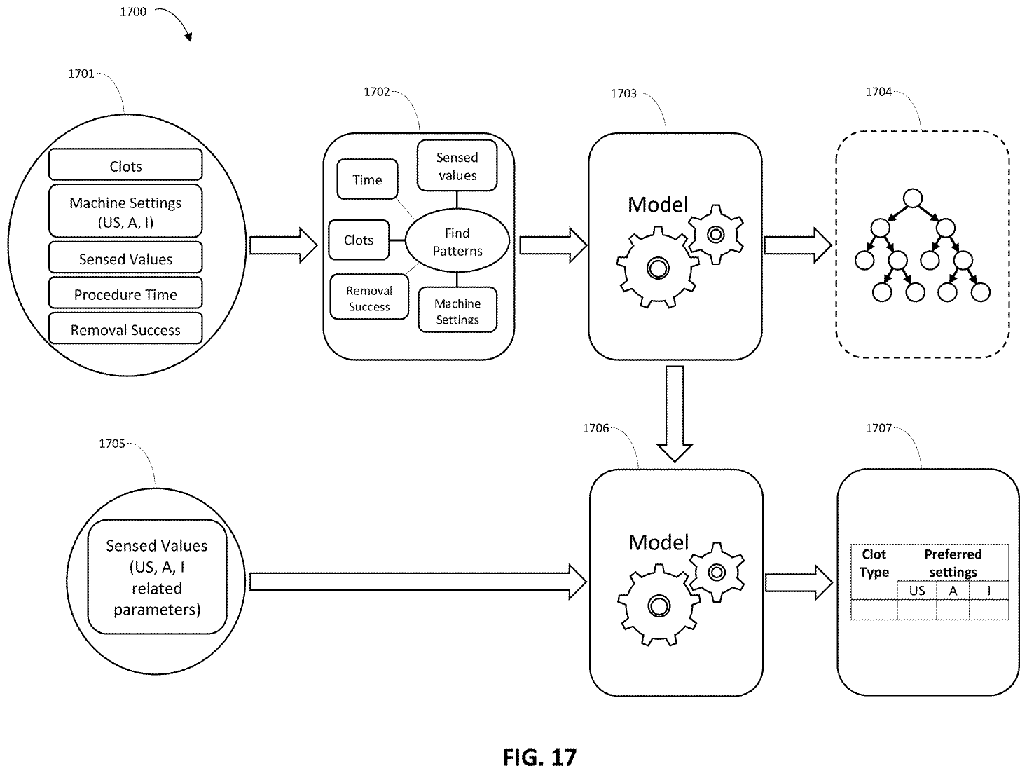

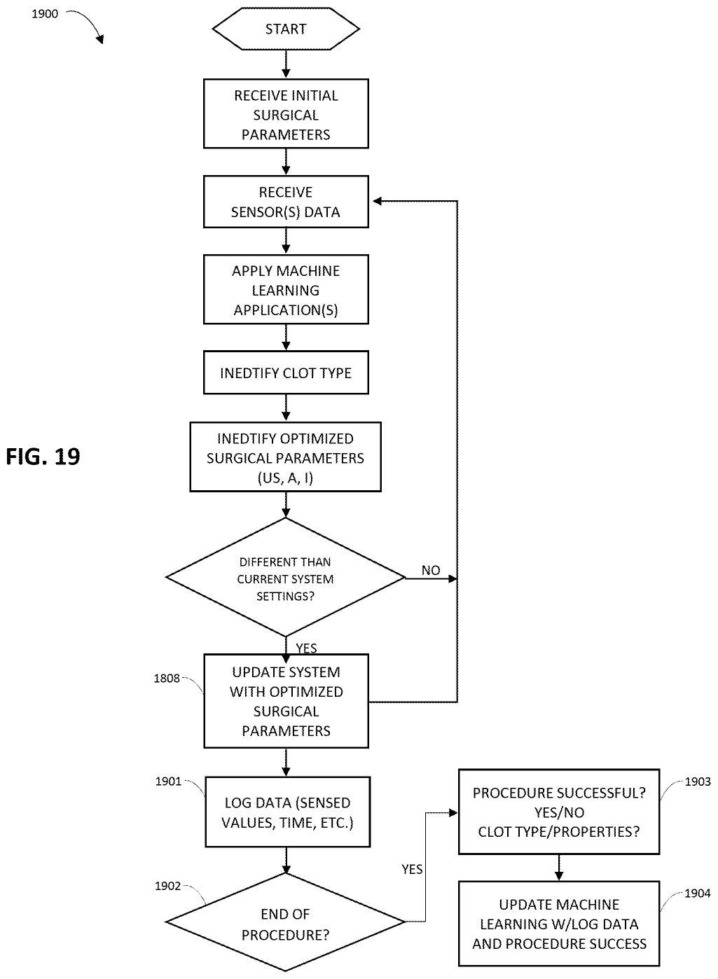

An example of a predictive machine learning application is illustrated in FIG. 17. The machine learning application 1700 is trained to learn, for example, different sensed parameter values associated with clot types and predict preferred/optimized system parameters for revascularization and removal of the specific clot. In this particular example, the machine learning application receives information 1701 on, but not limited to, clot types/properties, machine settings, sensed values (such as ultrasound and/or aspiration and/or irrigation related parameters as described above), procedure time and/or removal success. The data can be experimental data and/or data obtained from previous procedures. A machine learning training algorithm 1702 may be developed to train the machine learning to find patterns within the provided data. Based on the identified patterns, a machine learning model 1703 may be developed from which a pattern(s) database 1704 can be generated. With any new thrombectomy procedure, the sensed values associated with ultrasound (US) and/or Aspiration (A) and/or Irrigation (I) parameters (as described above) 1705 are input into the machine learning application(s), and the application(s) uses the built model(s) 1706 to determine clot type and optimized ultrasound (US) and/or aspiration (A) and/or irrigation (I) settings 1707 for the removal of the specific clot. In some embodiments, at the end of each procedure, the new collected data can be entered back into the machine learning application(s) to improve model(s)/predictability (FIG. 19). The machine learning application(s) can reside within a computing device internal and/or external to the system control, and one or more internal and/or external computing devices can be used to train the machine learning algorithm(s).

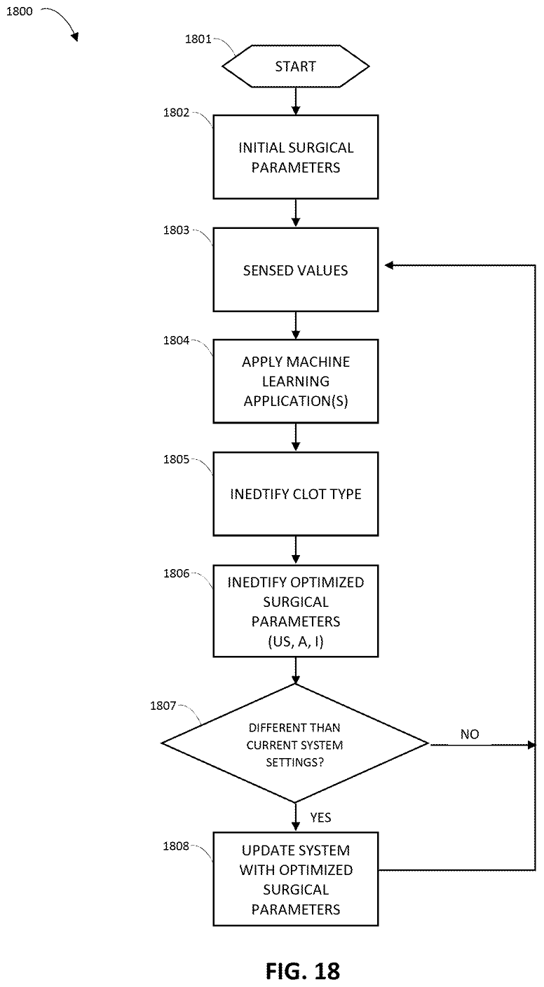

FIG. 18 is an exemplary flow chart 1800 describing a method for conducting a thrombectomy procedure that includes machine learning application(s). At the beginning of each new procedure at 1801, the system control may be set by internal algorithms and/or external source(s) (e.g. surgical staff, remotely via WiFi, etc.) with initial parameters at 1802. The input may be provided by an input device which may comprise a keyboard or display device associated with the console 132 (FIG. 1), or a touch screen on the console, or an external source for example. The control system may further include a foot pedal 134 (FIG. 1) used by an operator/surgeon to activate and/or set machine parameters. Once the procedure starts, the surgical system/machine is set with initial settings, and the surgical system/machine may be activated by any suitable means, the different sensed parameters are sensed at 1803 and their values communicated to the machine learning application(s) at 1804. The machine learning application(s) determines the clot type at 1805 and optimized ultrasound (US) and/or Aspiration (A) and/or Irrigation (I) system settings at 1806 for the identified type of clot. The optimized setting(s) can include, but not limited to, one or more values related to ultrasound power and/or frequency, aspiration levels and/or speed, and/or irrigation rates. The predicted optimized parameters are compared to current machine setting at 1807. If any of these values differ, the system may automatically be updated with new settings at 1808. In any case, the process 1803 to 1808 may repeat iteratively for as long as the procedure continues. This process can be executed continuously and/or at defined periods/intervals of time.

Further, a method for conducting a thrombectomy procedure that includes machine learning application(s) can also include processes where collected data can be entered back into the machine learning application(s) to improve model(s)/predictability. An exemplary embodiment is shown in the flow chart described in FIG. 19. With reference to FIG. 19, every time the system surgical parameters are updated at 1808, surgical data such as, but not limited to, sensed values and time, can be logged into a database/table at 1901. At the end of each procedure at 1902 or at a later stage, the surgeon defines if the procedure was successful at 1903. Successful procedure can be described by yes/no, and/or by a defined scale from say 1 to 10, and/or by the procedure time, and/or by outcome of the thrombectomy procedure as defined for example by mTICI and/or mRS scores, and/or by any other criteria and/or any combination thereof. Type/properties of clot removed during the surgical procedure can also be collected at the end of the surgery at 1903. Clot type/properties can be defined for example by a defined scale/score, and/or fibrin/platelet content and/or by pathology examination of removed clot. Following 1903, the log data at 1901 and procedure success data and/or clot data may be input back into the machine learning trained data and can be used to continue improvement of its model(s) and predictions.

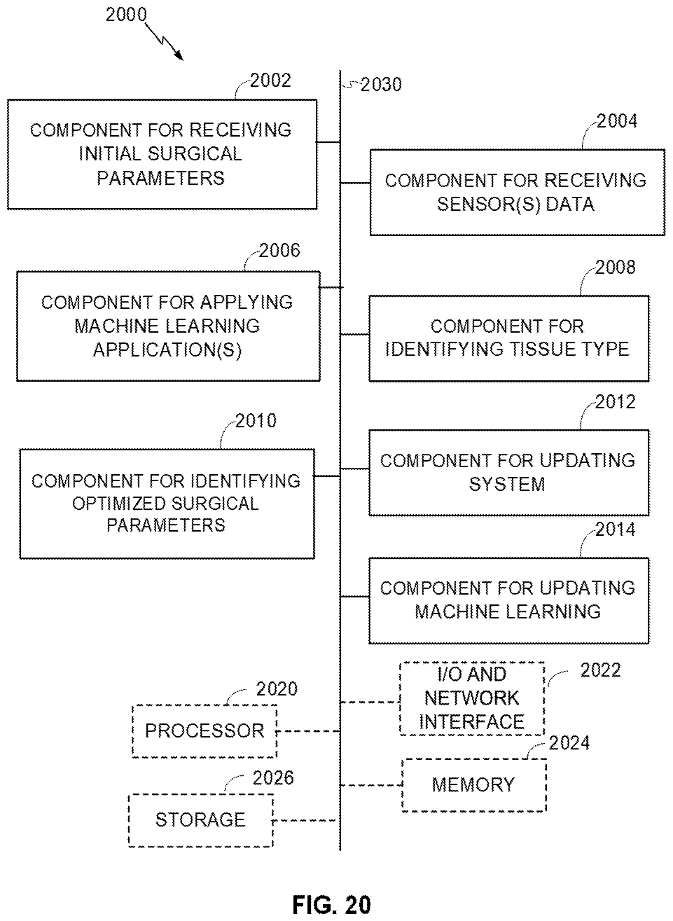

FIG. 20 illustrates a conceptual block diagram illustrating components of a system 2000 for determining surgical system settings during a surgical procedure as described herein, according to one embodiment. As depicted, the system 2000 may include functional blocks that can represent functions implemented by a processor, software, or combination thereof (e.g., firmware).

As illustrated in FIG. 20, the system 2000 may comprise an electrical component 2002 for receiving surgical parameters. The component 2002 may be, or may include, a means for said receiving. Said means may include the processor 2020 coupled to the memory 2024, storage 2026 which may store the database, and to the input/output and network interface 2022, the processor executing an algorithm based on program instructions stored in the memory. Such algorithm may include a sequence of more detailed operations, for example, as described in connection with FIGS. 13, 14, 18 and 19 above.

The system 2000 may further comprise an electrical component 2004 for receiving sensor(s) data. The component 2004 may be, or may include, a means for said receiving. Said means may include the processor 2020 coupled to the memory 2024, storage 2026 which may store the database, and to the input/output and network interface 2022, the processor executing an algorithm based on program instructions stored in the memory. Such algorithm may include a sequence of more detailed operations, for example, as described in connection with FIGS. 13, 14, 18 and 19 above.

The system 2000 may further comprise an electrical component 2006 for applying machine learning application(s). The component 2006 may be, or may include, a means for said applying. Said means may include the processor 2020 coupled to the memory 2024, storage 2026 which may store the database, and to the input/output and network interface 2022, the processor executing an algorithm based on program instructions stored in the memory. Such algorithm may include a sequence of more detailed operations, for example, as described in connection with FIGS. 13, 14, 18 and 19 above.

The system 2000 may further comprise an electrical component 2008 for identifying tissue type. The component 2008 may be, or may include, a means for said identifying. Said means may include the processor 2020 coupled to the memory 2024, storage 2026 which may store the database, and to the input/output and network interface 2022, the processor executing an algorithm based on program instructions stored in the memory. Such algorithm may include a sequence of more detailed operations, for example, as described in connection with FIGS. 4, 5, 12 and 13 above.

The system 2000 may further comprise an electrical component 2010 for identifying optimized surgical parameters. The component 2010 may be, or may include, a means for said identifying. Said means may include the processor 2020 coupled to the memory 2024, storage 2026 which may store the database, and to the input/output and network interface 2022, the processor executing an algorithm based on program instructions stored in the memory. Such algorithm may include a sequence of more detailed operations, for example, as described in connection with FIGS. 13, 14, 18 and 19 above.

The system 2000 may further comprise an electrical component 2012 for updating system data. The component 2004 may be, or may include, a means for said updating. Said means may include the processor 2020 coupled to the memory 2024, storage 2026 which may store the database, and to the input/output and network interface 2022, the processor executing an algorithm based on program instructions stored in the memory. Such algorithm may include a sequence of more detailed operations, for example, as described in connection with FIGS. 13, 14, 18 and 19 above.

The system 2000 may further comprise an electrical component 2014 for updating machine learning data. The component 2014 may be, or may include, a means for said receiving. Said means may include the processor 2020 coupled to the memory 2024, storage 2026 which may store the database, and to the input/output and network interface 2022, the processor executing an algorithm based on program instructions stored in the memory. Such algorithm may include a sequence of more detailed operations, for example, as described in connection with FIGS. 13, 14, 18 and 19 above.

While exemplary embodiments of the apparatus and methods are described above, it is to be understood that the above description is illustrative only and it is not intended that these embodiments describe all possible forms of the invention or limit the invention to the particular forms disclosed. Rather, the words used in the specification are words of description rather than limitation, and it is understood that various changes may be made without departing from the spirit and scope of the invention.

Although this disclosure describes specific applications of sensing and machine learning capabilities for thrombectomy in accordance with the present invention for the purpose of illustrating the manner in which the invention may be used to advantage, it should be appreciated that the invention is not limited thereto. Further, the invention illustratively disclosed herein suitably may be practiced in the absence of any element which is not specifically disclosed herein. The methods and embodiments of the present invention have specifically been discussed with reference to thrombectomy. However, the methods and embodiments have equal application to other medical arts, including those in which are used for removal of any tissue structure. Accordingly, any and all modifications, variations or equivalent arrangements which may occur to those skilled in the art, should be considered to be within the scope of the present invention.

What is claimed is:

1. A system for removing a selected portion of tissue structure from a part of a human body, the system comprising:
a catheter having a tubular body comprising an outer sheath and an inner core;
a hub assembly connected at a proximal end of the catheter for coupling to a control system;
a transducer;
a horn coupled to the transducer; and
a hollow needle, having an inner lumen, coupled directly or indirectly to the horn, wherein the hollow needle includes a distal cutting tip to fragment and/or emulsify the selected portion of tissue structure.

2. The system of claim 1, wherein the transducer is coupled to a distal end of one of the inner core or the outer sheath.

3. The system of claim 1, wherein the transducer is coupled to the hub assembly and the inner core.

4. The system of claim 1 further comprises a guidewire sized to pass longitudinally through an entire length of the catheter and an inner lumen of the hollow needle, the guidewire includes one or more deployable surgical assisting elements.

5. The system of claim 1, wherein the hub assembly comprises a sealed port for a guidewire, an aspiration port, an irrigation port and a drive circuitry port.

6. The system of claim 1, wherein the control system comprises an associated drive circuitry configured to provide a variable frequency alternating current to drive or excite the transducer at a select operating frequency and causes oscillation of the horn and vibration of the hollow needle.

7. The system of claim 6, wherein the control system is configured to vary the vibration of the hollow needle, to increase or decrease a mechanical cutting performance and/or the cavitational-induced performance of the hollow needle.

8. The system of claim 6, wherein the control system is configured to permit selection of an operating frequency, an aspiration rate and an irrigation rate.

9. The system of claim 6, wherein the control system receives inputs from at least one of an operator, one or more applications including machine learning application(s) and an external source.

10. The system of claim 1, wherein the horn and the transducer take the form of one of solid rods, disks, or a plurality of smaller elements.

11. The system of claim 4, wherein a deployable surgical assisting element has a first condition wherein the surgical assisting element is retracted/collapsed and a second condition wherein the surgical assisting element is expanded/open spanning substantially an entire lumen of a vessel.

12. The system of claim 1 further comprises one or more of pressure sensors, flow sensors, accelerometers and displacement sensors.

13. The system of claim 12, wherein the one or more sensors measure one or more parameters including ultrasound characteristics, vacuum pump speed, pressure levels, suction level and irrigation flow.

14. The system of claim 13, wherein the control system is further configured to:
receive one or more initial surgical parameters;
receive data from the one or more sensors;
apply a machine learning application to the one or more initial surgical parameters and the data from the one or more sensors;
identify a tissue type; and
identify optimized surgical parameters.

15. A method for removing a selected portion of tissue structure from a part of a human body, the method comprising the steps of:
receiving one or more surgical parameters;
receiving data from one or more sensors;
applying a machine learning application to the one or more initial surgical parameters and the data from the one or more sensors;
identifying a tissue type; and
identifying one or more optimized surgical parameters.

16. The method of claim 15 further comprises determining one or more preferred system parameter settings for a surgical system based on the one or more optimized surgical parameters.

17. The method of claim 16 further comprises at least one of automatically adjusting and suggesting system parameters of the surgical system during a surgical procedure based on the determined one or more the preferred system parameter settings.

18. The method of claim 15, wherein the one or more sensors comprise one or more of pressure sensors, flow sensors, optical sensors, accelerometers, temperature, and displacement sensors.

19. The method of claim 15, wherein the one or more optimized surgical parameters are used to train the machine learning application.

20. The method of claim 17, wherein the surgical system further comprises:
a catheter having a tubular body comprising an outer sheath and an inner core;
a hub assembly connected at a proximal end of the catheter for coupling to a control system;
a transducer;
a horn coupled to the transducer; and
a hollow needle, having an inner lumen, coupled directly or indirectly to the horn, wherein the hollow needle includes a distal cutting tip to fragment and/or emulsify the selected portion of tissue structure.
